(19) United States
(12) Patent Application Publication
(10) Pub. No.: US 2007/0208376 A1
(43) Pub. Date: Sep. 6, 2007

(54) Occlusion device with internal fastener

(76) Inventor: Jian Meng, Beijing (CN)

Correspondence Address: VAN PELT, YI & JAMES LLP, 10050 N. FOOTHILL BLVD #200, CUPERTINO, CA, 95014, US

(21) Appl. No.: 11/654,831

(22) Filed: Jan. 17, 2007

(30) Foreign Application Priority Data
Mar. 3, 2006 (CN) 200610073771.9

(51) Int. Cl. A61B 17/08

(52) U.S. Cl. 606/213

(57) ABSTRACT

An occlusion device comprises a plurality of wires made of shape memory material, braided to form an expanded shape having a first end and a second end, a first fastener coupled to the plurality of wires at the first end, and a second fastener coupled to the plurality of wires at the second end, wherein the first fasteners is placed in the interior of the device. Another occlusion device comprises a first disk shaped portion made of shape memory material, a first fastener configured to fasten one end of the first disk shaped portion, a second disk shaped portion made of shape memory material, a second fastener configured to fasten one end of the second disk shaped portion, and a tension member connecting the first portion and the second portion, wherein the first fasteners is placed in the interior of the device.

### CROSS REFERENCE TO OTHER APPLICATIONS

This application claims priority to Chinese Patent Application No. 200610073771.9 entitled MEDICAL OCCLUSION DEVICE AND ITS MANUFACTURING filed Apr. 3, 2006 which is incorporated herein by reference for all purposes.

### BACKGROUND OF THE INVENTION

Minimally invasive/noninvasive treatment of cardiovascular diseases is an important area of development for modern medicine. Since the introduction of interventional treatments for cardiovascular diseases in 1964 by Dotter, there have been many new procedures and devices developed for non-surgical treatments. A number of occlusion devices have been developed for stopping blood flow through a blood vessel or heart chamber.

The occlusion device in existence today is typically constructed of braided wires made of shape memory metal (e.g. a nickel-titanium alloy commercialized under the trade name Nitinol). The wires are heat treated to form a preconfigured, remembered shape. Post heat treatment, if device becomes distorted, it will to return to the remembered shape when it is released. FIG. 1A is a side view illustrating a typical occlusion device. Device 100 is constructed using a plurality of braided Nitinol wires 102. The diameter of the wires may vary for different devices. Wires having diameters of 0.02-0.05 mm are commonly used. The number of wires used depends on factors such as the diameter of the wires. In some devices, sixteen to thirty-two pieces of wires are braided together to form the device.

The ends of the wires are fastened to radiopaque bands 104a and 104b that are made of materials such as platinum-iridium al By, titanium, platinum, or gold. The diameters of the bands and the distance between the bands vary depending on the application. The bottom band 104b forms a screw, allowing the device to be attached to the distal end of a delivery cable 108. Device 100 is shown here in a collapsed configuration. The device is elongated along its longitudinal axis so that it may be placed within a French size catheter 110. During deployment, the catheter is placed in a blood vessel or an organ of the patient's body, and the device with the delivery cable attached is introduced through the catheter. The delivery cable pushes the device to advance the device along the catheter, positioning the device at a desired deployment site.

FIG. 1B is a sectional view illustrating a partially deployed occlusion device of FIG. 1A. As shown, the delivery cable has forced band 104a and a portion of the device assembly to exit the distal end 112 of catheter 110. FIG. 1C is a sectional view illustrating a fully deployed occlusion device of FIG. 1A. As shown, device 100 has fully exited the catheter, and the delivery cable is disengaged from the device. The wires have extended to their remembered shape (also referred to as the relaxed shape). The resulting remembered shape of device 100 has two annular wire mesh disks 120a and 120b, which are connected by a cylindrical segment 124 having a reduced diameter. The device is implanted in the patient's heart to treat septal defects.

Although occlusion devices similar to device 100 have proven to be useful for minimally invasive and non-invasive cardiovascular treatments, the inventor has recognized that several issues remain in practice. For example, the fastening bands of the typical occlusion device protrude from the wire mesh disks' surface, preventing tissues from growing around the bands and potentially causing thrombosis. FIG. 2A is a sectional view illustrating an improved occlusion device design. In this sectional diagram, only a limited number of wires are shown for purposes of illustration. Each of the wire mesh disks of device 200 has a recessed portion that extends inward from the outer surface of the disks. Fasteners 202a and 202b, which are attached to the mesh fabric and positioned on the outer surface of the device, are contained within the recessed portions and positioned substantially below the outer surface of the disks. Although this design improves the safety of the device by reducing the likelihood of thrombus formation on the surface of the device, thrombus can still form because the surfaces of the disks are not very smooth.

The inventor recognizes that another problem associated with using an occlusion device for correcting a ventricular septal defect (VSD) in a patient's heart is the pressure and friction against the heart wall. FIG. 2B is a side view illustrating an occlusion device placed in a patient's heart. In the example shown, device 250 is placed across the interventricular septum to close a VSD. A nerve called His branch is located in this region. The cylindrical portion of the occlusion device tends to exert pressure on the septum, and can pinch the nerve, thereby causing edema. Further, the friction between the device and the heart tissues can damage or even sever the nerve, causing atrioventricular block and arrhythmia.

The inventor further recognizes that the metal wires used to construct the device are in direct contact with body tissues such as blood vessels and heart chamber walls. After the device is implanted, the metal wires can rub against the body tissues, and the friction can cause tissue damage. The danger of tissue damage due to friction is particularly great when the device is implanted in the heart.

The inventor further recognizes that the metallic material of the wires tends to slow new tissue growth on the surface of the device. Since the device is usually implanted for long periods of time and cannot be absorbed by the body or assimilated by the body tissues, it can cause various undesirable side effects, some of which are life threatening.

The inventor further recognizes that the disk portions of the device typically have a sharp side profile, which can cause tissue damage.

The inventor recognizes that it would be desirable to have an occlusion device that can reduce the risks of thrombosis. It would also be useful to have a device that could promote tissue growth. It would also be useful to have a device that could lessen the pressure to the tissues and reduce tissue damage.

### BRIEF DESCRIPTION OF THE DRAWINGS

Various embodiments of the invention are disclosed in the following detailed description and the accompanying drawings.

FIG. 1A is a side view illustrating a typical occlusion device.

FIG. 1B is a sectional view illustrating a partially deployed occlusion device of FIG. 1A.

FIG. 1C is a sectional view illustrating a fully deployed occlusion device of FIG. 1A.

FIG. 2A is a sectional view illustrating an improved occlusion device design.

FIG. 2B is a side view illustrating an occlusion device placed in a patient's heart.

FIG. 3A is a side view of an embodiment of an occlusion device.

FIG. 3B is a side view illustrating an embodiment of an occlusion device in its compressed state.

FIG. 3C is a side view showing another embodiment of an occlusion device.

FIG. 4A is a 3D hidden line view of a fastener embodiment.

FIG. 4B is a 3D hidden line view of another fastener embodiment.

FIGS. 5A-5C are diagrams illustrating one way of manufacturing an occlusion device.

FIG. 6A is a side view of an occlusion device embodiment.

FIG. 6B is a side view of another embodiment of an occlusion device that includes a spring.

FIG. 7A is a side view diagram of an embodiment of an occlusion device that includes a patch.

FIGS. 7B-7D are side view diagrams illustrating additional occlusion device embodiments with one or more patches.

FIG. 8A is a side view diagram illustrating the contour of an embodiment of an occlusion device having enlarged, bulbous disk edges.

FIG. 8B is a diagram illustrating an occlusion device embodiment having a tension member, a bulbous edge profile, and internal fasteners.

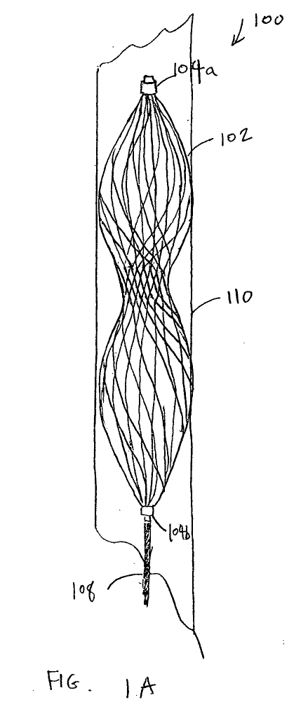

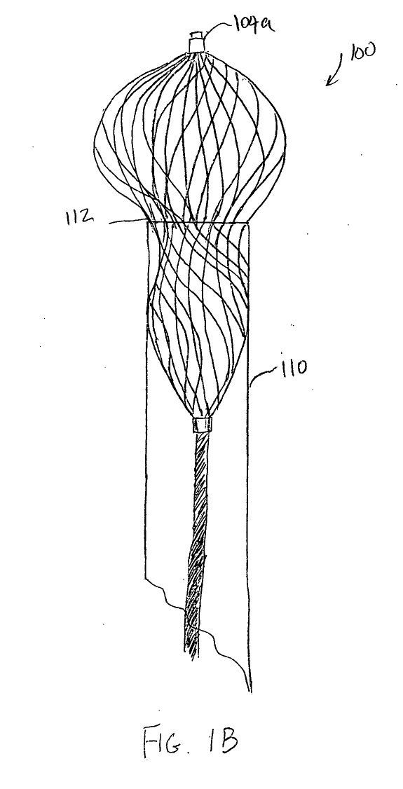

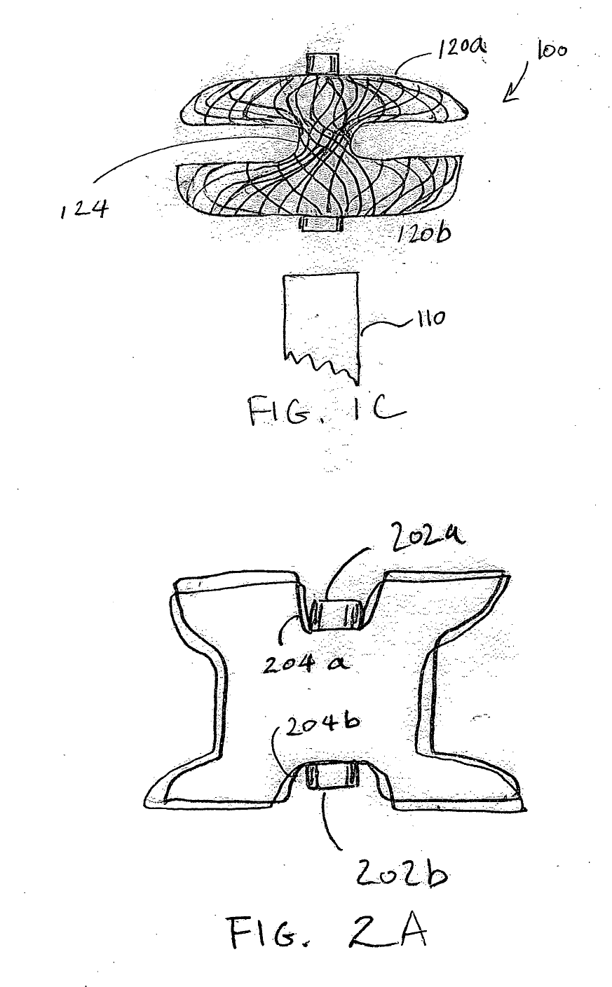

### DETAILED DESCRIPTION

The invention can be implemented in numerous ways, including as a process, an apparatus, a system, a composition of matter. In this specification, these implementations, or any other form that the invention may take, may be referred to as techniques. In general, the order of the steps of disclosed processes may be altered within the scope of the invention.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

An occlusion device for use in occluding an abnormal opening in a patient's body is described. In some embodiments, the occlusion device includes two disk shaped portions made of shape memory material and a tension member, such as a spring, that connects the two portions. In some embodiments, the occlusion device includes braided wires that form an expanded shape having a first end and a second end, and fasteners coupled to two ends of the braided wires. At least one of the fasteners is placed within the interior of the expanded shape. In some embodiments, the occlusion device includes a non-metal covering. In some embodiments, the disk portions of the occlusion device have a bulbous shape.

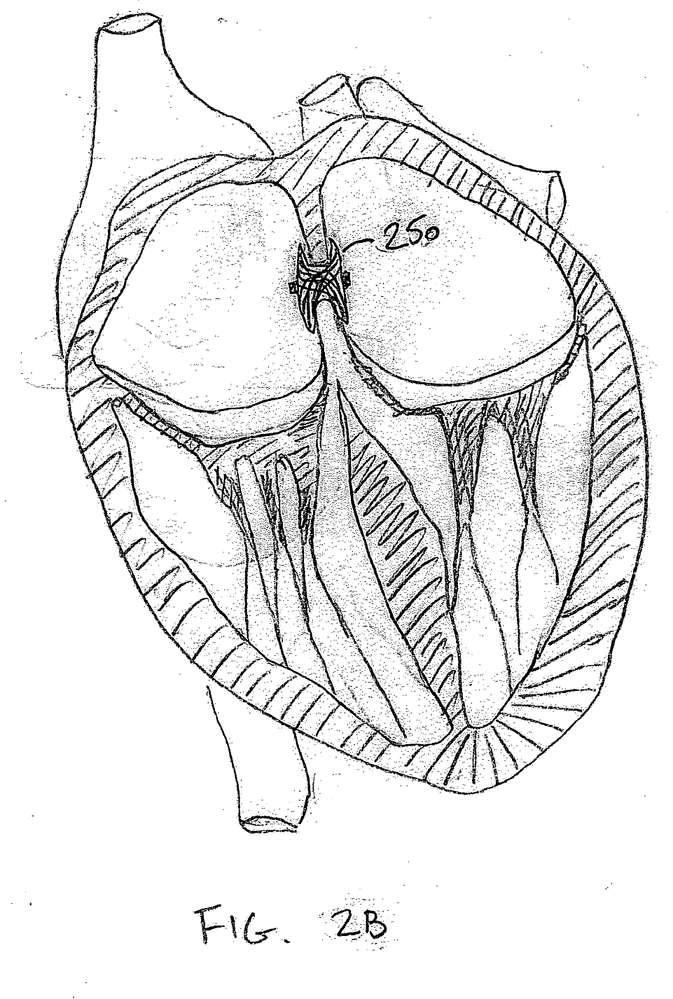

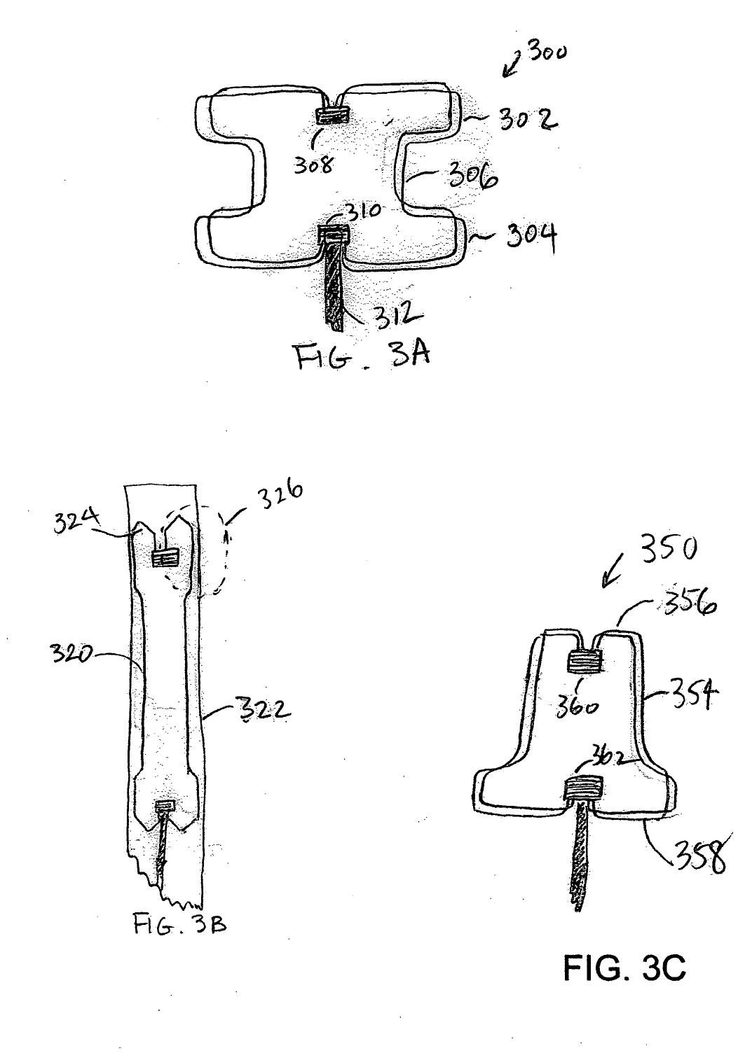

FIG. 3A is a side view of an embodiment of an occlusion device. Device 300 shown in this example is suitable for treatment of VSD. The device is shown in its relaxed state, in its remembered shape. The device is formed by braiding or winding a plurality of wires made of shape memory material. Although metallic wires such as wires made of NiTi alloy are discussed in detail in the following specification, other non-metallic material such as plastic polymers may be used. For purposes of illustration, a few wires are shown in the diagram, even though a greater density of wires are used in practice. The wires form two expanded disk shaped portions 302 and 304, which are connected by a waist portion 306. The waist portion has a cross sectional diameter that is less than the expanded outer diameter of either disks. The diameters of the disks and the waist portion are chosen to be somewhat greater than the defect. For example, for a defect that is 24-25 mm in diameter, the diameter of the waist portion of the device is chosen to be about 2-4 mm greater, and diameter of the disk portions are 7-8 mm greater.

The ends of the wires forming the disk portions are attached to fasteners 308 and 310, which are made of radiopaque material. In this example, both fasteners are placed substantially within the interior space of the device so that they do not protrude from the surface of the device. Placing the fasteners in the interior space of the device provides a smoother, flatter outer surface, which promotes the growth of the new endothelial layer and reduces the likelihood of thrombus formation. It also prevents the fasteners from protruding into the wall of the blood vessel or heart and causing tissue damage. Fastener 310 is shown to be configured to engage the distal end of a delivery cable 312. The delivery cable assists in placing the device during deployment, and is removed once the device is in position. In some embodiments, proximal fastener 310 is placed on the outside of the device.

FIG. 3B is a side view illustrating an embodiment of an occlusion device in its compressed state. Device 320 is compressed to fit within the lumen of catheter 322. Where a piece of wire is folded, the tension in the wire tends to give the folded corner region a round shape. For example, near region 326 where the wires are folded over the fastener, without any special treatment, the wires naturally tend to form a round, bulging shape. This round profile can increase the profile diameter of the occlusion device, making it difficult to fit the device within the lumen of small-diameter sheaths. To better accommodate the size and shape of the sheath, wires near the fastener are biased to conform to the fastener, so that the wires are closer to the fastener than they would otherwise be. This may be done, for example, by bending the wires to form an angle 324. The biased wires have reduced tension in the folded region, and the profile of the fold is smaller.

FIG. 3C is a side view showing another embodiment of an occlusion device. Device 350 shown in this example is suitable for use in patent ductus arteriosus (PDA) treatment. The relaxed shape of the device is defined by a plurality of braided wires, where only a few wires are shown to illustrate the shape of the device. The fabric forms a hat shape having a tapered cylindrical portion 354, a smaller end 356, and a larger, disk shaped end 358. Similar to device 300 shown previously, fasteners 360 and 362 are positioned to be substantially within the device.

FIG. 4A is a 3D hidden line view of a fastener embodiment. In the diagram shown, fastener 470 has the shape of a hollow cylinder. Portions of some of the wires are shown. Wires 474 are attached to the bottom edge of the cylinder and are folded outward, enclosing the top edge of the cylinder inside the space defined by the wires. A set of internal threads are formed on the interior of the cylinder to engage the external threads on the distal end 476 of a delivery cable 472.

FIG. 4B is a 3D hidden line view of another fastener embodiment. In this example, fastener 480 includes an inner cylindrical portion 482. A set of external threads is formed on the cylindrical portion. The distal end of a delivery cable 484 includes a hollow cylindrical portion that has a set of internal threads. The external threads of the fastener are configured to engage the internal threads of the delivery cable. The fastener further includes an outer portion 486, shaped like a hollow cylinder, to which the wires are attached. Other fastener configurations are also possible.

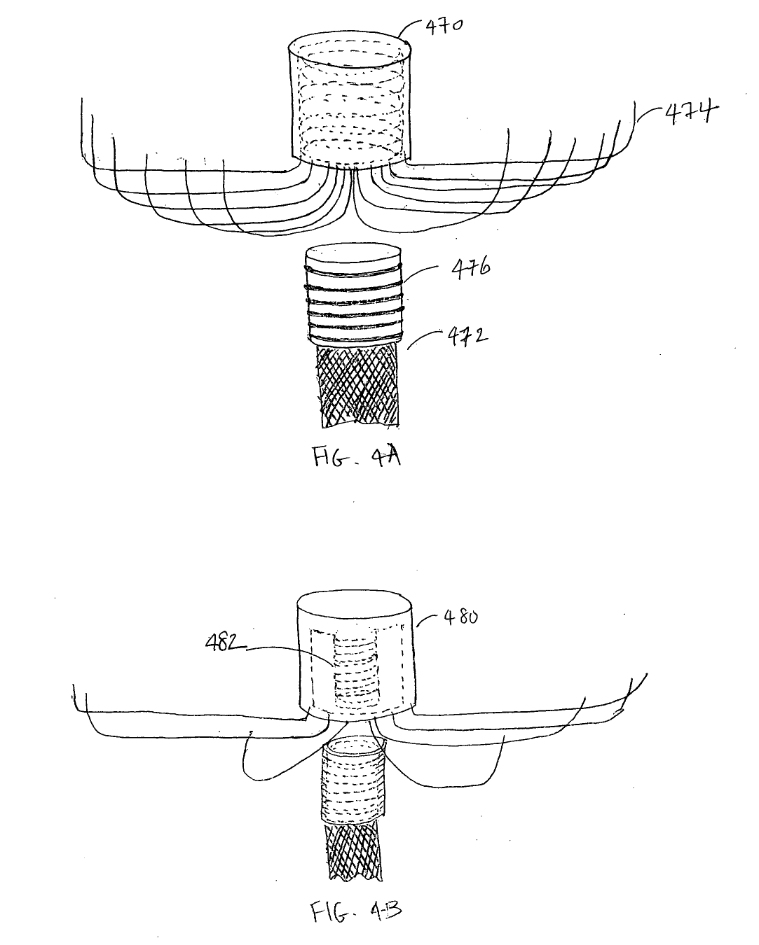

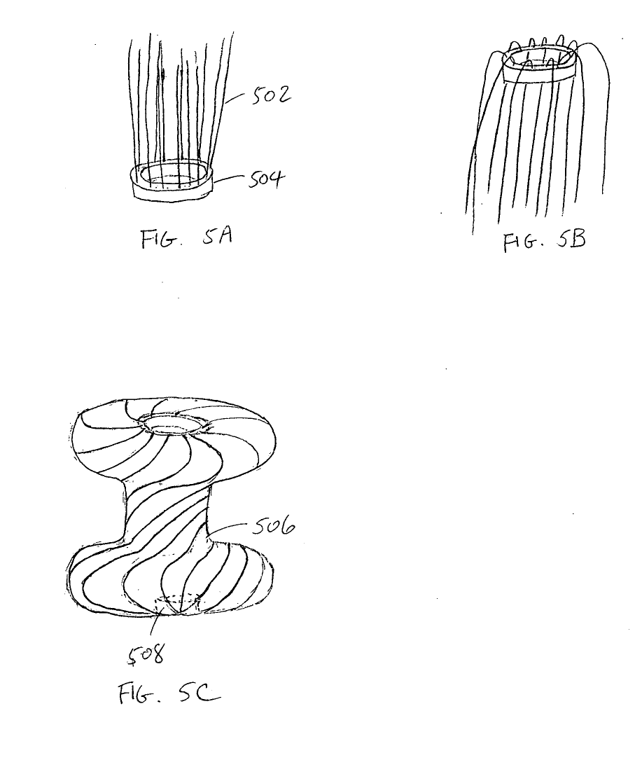

FIGS. 5A-5C are diagrams illustrating one way of manufacturing an occlusion device. In FIG. 5A, a plurality of wires 502 are provided. The number of wires depends on device requirement. The wires are evenly spaced and attached to a circular fastener 504 on one end. The wires may be welded, clamped, soldered, brazed, or otherwise attached firmly to one edge of the fastener. In FIG. 5B, the wires are folded over to surround the fastener, placing the fastener inside the group of wires.

In FIG. 5C, the wire-fastener assembly is fitted over a mold 506, which has approximately the desired shape and dimensions of the fully deployed occlusion device. Fastener 504 is seated within a recessed portion on the top side of the mold. The wires are braided or wound along the outer surface of the mold, following appropriate paths and directions to form the desired pattern. The ends of the wires are attached to a second fastener 508 (shown in dash). In some embodiments, fastener 508 is a component similar to 470 or 480 of FIGS. 4A and 4B. It is initially placed in an indentation of the mold. The ends of the wires are trimmed if necessary, and attached to the outer edge of fastener 508.

The mold is made of a material that can withstand the heat treatment without deforming, and can be removed without significantly changing the properties of the formed occlusion device. In some embodiments, the mold is made of plastic or resin material that can be chemically dissolved. In some embodiments, the mold is made of glass or ceramic material that can be shattered into small pieces that are then extracted from the opening of the occlusion device.

The whole assembly is heat treated to set the remembered shape of the occlusion device. Parameters of the heat treatment such as temperature and duration depend on the materials used and may vary in different processes. The mold is then removed using a technique appropriate for the mold material. For example, the mold may be shattered mechanically or dissolve chemically. This method produces an occlusion device having two fasteners that lie within the device. Alternatively, a mold with only a single indentation for receiving a single one of the fasteners may be used. In which case the wires can be attached to the second fastener from the opposite direction, leaving the second fastener on the outside of the shape defined by the braided wire.

The method described above is one example of how to manufacture an occlusion device. Additional steps or alternative steps are possible. A number of alternative techniques exist. For example, a pre-fabricated wire mesh made of shape memory material may be used instead of individual strands of wires. In some embodiments, the device is generated according to the following steps: a tube made of the pre-fabricated wire mesh is provided. A first fastener is used to fasten one side of the tube. The tube is turned inside out, thus placing the fastener in the interior space of the device. A mold is placed inside the tube to shape the device. After heat treatment, the mold is removed. A second fastener is used to close the opening of the device. In some embodiments, a flat piece of wire mesh fabric is provided. Two fasteners are placed on two opposite sides of the wire mesh fabric piece. A mold is placed inside the mesh fabric, and the wire mesh fabric is folded towards the middle to conform to the shape of the mold. The wire mesh fabric is welded in the places necessary. The assembly is heat treated, and the mold is removed.

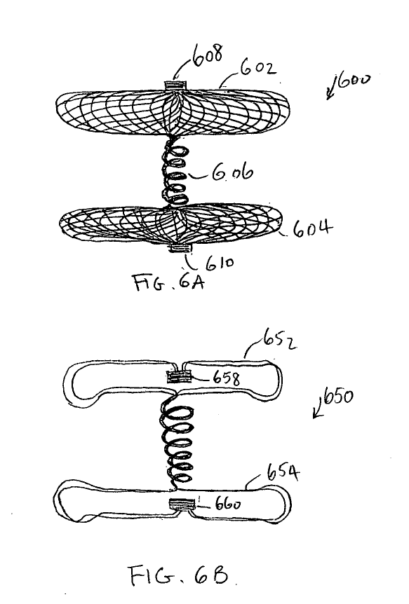

To prevent damage to heart tissues and nerve, some embodiments of the occlusion device use a tension member, such as a spring, to connect the disk portions. The tension exerted by the tension member hold the disk portions in place to occlude the defect. FIG. 6A is a side view of an occlusion device embodiment. In the example shown, the occlusion device includes two disks 602 and 604 that are formed using braided wire. A spring 606 connects the two disks. The spring engages each disk substantially in the disk center. The extended length of the spring is approximately the length of the defect to be occluded. For example, for a VSD occlusion device, the length is approximately the thickness of the septal wall. The diameter of the spring is substantially less than the diameter of the defect, thus avoiding contact with the septal wall. For example, for a defect of 24-25 mm in diameter, the tension member can have a diameter that is 2-20 mm. Since this arrangement significantly reduces friction between the heart muscle and the device, the risks of edema and arrhythmia are greatly reduced.

In some embodiments, the disk portions and the spring are formed separately. The spring is then attached to the centers of the disks. In some embodiments, the spring is formed using the same wires that are used to construct the disk portions. To make the occlusion device, a plurality of wires are provided. The wires are fastened by fastener 608 on one end. The free, unfastened portions of the wires are braided to form disk 602. A mold having the desired disk shape is used to aid the braiding process. On the end of the disk opposite the fastener, the wires are gathered into a single bundle. The wires are optionally braided together to make the bundle more stable structurally. The wire bundle is wound to form spring 606. Once the desired length is achieved, the wires are separated and braided to form a second disk 604. A second disk mold is used to facilitate the formation of the second braided disk. The ends of the wires are collected and fastened to fastener 610.

FIG. 6B is a side view of another embodiment of an occlusion device that includes a spring. A limited number of wires are shown for purposes of clarity. In the example shown, the ends of wires forming disks 652 and 654 are attached to fasteners 658 and 660. The fasteners are positioned within the interior space of the device, providing a smoother and flatter outer disk surface and promoting better endothelial layer growth. Device 650 shown in this example can be constructed using techniques similar to the techniques used to construct device 600, except that once the wires are attached to the fastener, they are turned inside out to place the fastener in the interior space of the disk.

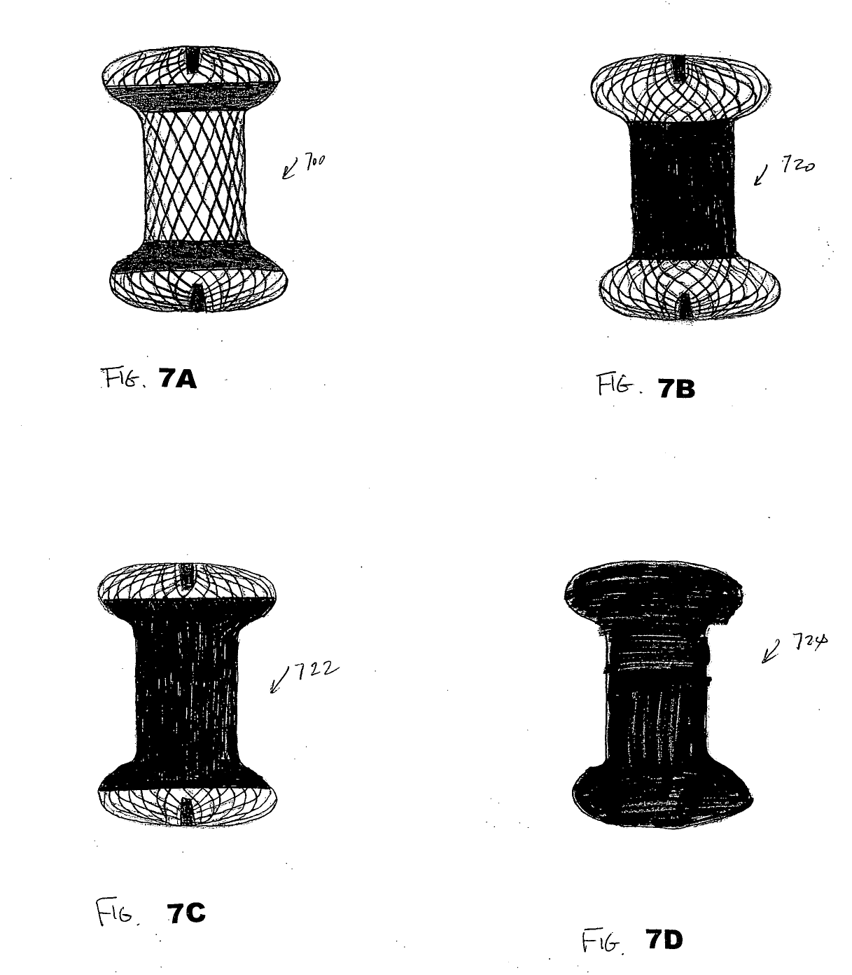

Sometimes the wire fabric of the device can slow down the tissue development. Some occlusion device embodiments address this issue by covering at least a portion of the device surface with a patch. FIG. 7A is a side view diagram of an embodiment of an occlusion device that includes a patch. In the example shown, parts of the outer surfaces of device 700's disk portions are covered with patches made of a soft material. The patches are attached to areas that are in contact with the patient's body tissues when the device is deployed. The patches make the surface of the device smoother and promote the growth of the endothelial layer, thus reducing the friction between the occlusion device and the body tissues and decreasing the risk of tissue damage to the patient. FIGS. 7B-7D are side view diagrams illustrating additional occlusion device embodiments with one or more patches. For example, device 720 includes a patch in its waist portion. Device 740 includes a patch in its waist portion and parts of the disk portions. Nearly all of Device 724 is covered by the patch material, except for the area near the fastener that is attached to the delivery cable during delivery. Suitable locations for the patch include areas of the device that are in contact with the patient's heart tissues when the device is employed.

The patch can be made from a variety of materials, such as soft plastic material, biocompatible material, biodegradable material, bioabsorbent material that can be absorbed by the body, and/or biomedical material formed using cultured body tissues. Examples of the material include polyester, nylon, polyurethane, polylactic acid (PLA), polyglycolic acid (PGA), poly(lactic-co-glycolic acid) (PLGA), as well as other biomedical synthetic plastic and rubber material. In various embodiments, the patch is glued, wrapped, stitched, or otherwise adhered to the metal fabric material.

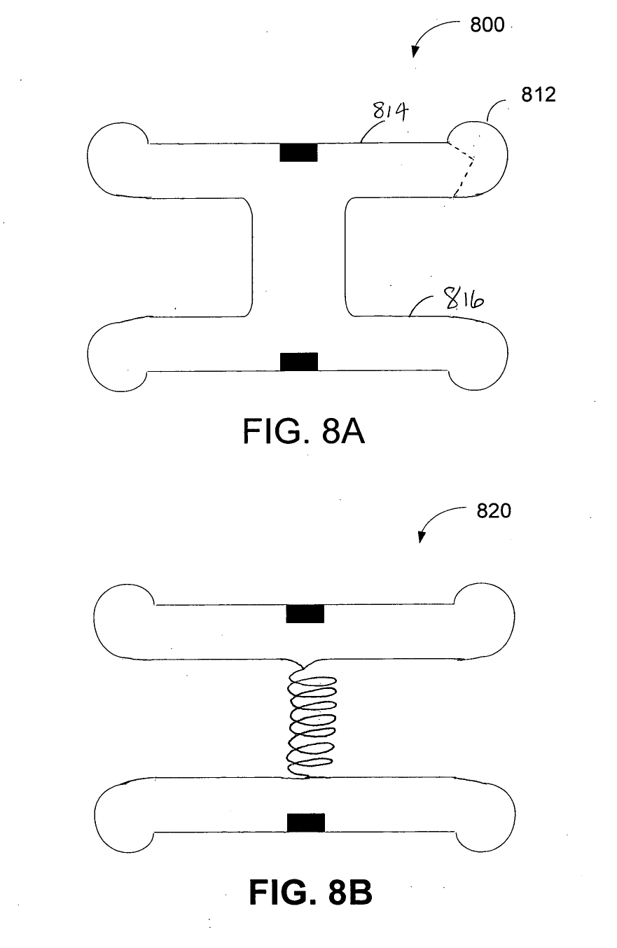

In some embodiments, to prevent the sharp edges of the disk portions of the occlusion device from causing tissue damage, the disk edges are arranged to have a bulbous profile. In other words, the edge portion of at least one disk is configured to have a smooth profile and form a bulge in the direction opposite of the disk center. FIG. 8A is a side view diagram illustrating the contour of an embodiment of an occlusion device having enlarged, bulbous disk edges. In this example, the edge portions of the disks have cross sectional areas that are wider than the rest of the disk portions. As shown, edge portion 812 approximately forms an arc having a central angle greater than 180°. Other geometry is possible as long as the edge portion of the disk is formed to have a smooth shape and a greater cross sectional diameter than the thickness of the disk. The bulbous profile prevents the edges of the expanded disk portions of the occlusion device from slicing into septal tissues and causing damage. Further, the wires forming each disk are arranged to bulge on disk side 814, which is not in direct contact with the septum when the device is deployed in the patient's heart. Disk side 816, which is in contact with the septum, is arranged to be relatively smooth and substantially free from pressure points where pressure may be concentrated and exerted on the septum. This arrangement allows more force to be exerted in the direction of the septum to more securely hold the device in place.

An occlusion device for use in occluding an abnormal opening in a patient's body has been described. In various embodiments, the occlusion device may have a tension member connecting two disk portions, one or more fasteners placed on the interior of the device, a non-metal cover, a disk edge profile that is bulbous. More than one of these aspects may be present in a single device. For example, a device with a tension member connecting the disk portions may also have one or more internal fasteners, a cover, and/or a bulbous edge profile. A device having one or more internal fasteners may include a cover, and/or have a bulbous edge profile. FIG. 8B is a diagram illustrating an occlusion device embodiment having a tension member, a bulbous edge profile, and internal fasteners. Other combinations are possible.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. An occlusion device comprising:
a plurality of wires made of shape memory material, braided to form an expanded shape having a first end and a second end;
a first fastener coupled to the plurality of wires at the first end; and
a second fastener coupled to the plurality of wires at the second end; wherein
the first fasteners is placed in the interior of the device.

2. An occlusion device as recited in claim 1, wherein the device is adapted to close a septal defect.

3. An occlusion device as recited in claim 1, wherein the first fastener is configured to be coupled to a delivery cable during delivery of the occlusion device.

4. An occlusion device as recited in claim 3, wherein the first fastener includes a set of internal threads configured to be coupled to the distal end of the delivery cable.

5. An occlusion device as recited in claim 3, wherein the first fastener includes an inner portion having a set of external threads configured to be coupled to the distal end of the delivery cable.

6. An occlusion device as recited in claim 1, wherein the second fastener is placed in the interior of the device.

7. An occlusion device as recited in claim 1, wherein the occlusion device is collapsible to form a collapsed configuration, and in the collapsed configuration the plurality of wires are biased to conform to the first fastener at the first end.

8. An occlusion device as recited in claim 1, further comprising a nonmetal patch attached to at least a portion of the occlusion device.

9. An occlusion device as recited in claim 1, wherein at least one of the ends is disk shaped and has an edge portion with a bulbous profile.

10. An occlusion device comprising:
a first disk shaped portion made of shape memory material;
a first fastener configured to fasten one end of the first disk shaped portion;
a second disk shaped portion made of shape memory material;
a second fastener configured to fasten one end of the second disk shaped portion; and
a tension member connecting the first portion and the second portion; wherein
the first fasteners is placed in the interior of the device.
